(19) United States
(12) Patent Application Publication
(10) Pub. No.: US 2021/0032644 A1
(43) Pub. Date: Feb. 4, 2021

(54) METHOD FOR INTRODUCING SUBSTANCE INTO PLANT CELL

(71) Applicants: JAPAN TOBACCO INC., Tokyo (JP); RIKEN, Wako-shi (JP)

(72) Inventors: Keiko MANNEN, Saitama (JP); Minami MATSUI, Saitama (JP); Takashi OKAMOTO, Saitama (JP); Norio KATO, Shizuoka (JP)

(73) Assignees: JAPAN TOBACCO INC., Tokyo (JP); RIKEN, Wako-shi (JP)

(21) Appl. No.: 16/943,913

(22) Filed: Jul. 30, 2020

(30) Foreign Application Priority Data
Jul. 31, 2019 (JP) 2019-141246

(51) Int. Cl. C12N 15/82

(52) U.S. Cl. CPC C12N 15/8207

(57) ABSTRACT

The present invention relates to a method for introducing a substance into a plant cell. The method of the present invention comprises introducing a substance into a plant cell present in the liquid phase using the particle gun method.

### TECHNICAL FIELD

The present invention relates to a method for introducing a substance into a plant cell, and to a substance-introduced plant cell.

### BACKGROUND ART

Transgenic technologies in plants such as the Agrobacterium method, the particle gun method, the electroporation method, and the polyethylene glycol method (PEG method) have been developed. In particular, development of transgenic technologies in monocotyledonous plants has been initially difficult, but its use has spread rapidly since the methods using Agrobacterium were developed for rice and maize in the 1990's. However, it is known that the efficiency of transformation greatly differs due to the influence of the difficulty of tissue culture depending on the species and variety, since the methods mostly need to undergo dedifferentiation and redifferentiation of plant tissues. For certain species and varieties, the efficiency of tissue culture is low, and transformed plants with reproducibility cannot be obtained. For example, B73, which is a very important strain for maize breeding, has been recognized as a typical difficult-to-culture variety, and it has been difficult to produce a transformation method thereof with reproducibility. Recently, methods for acquiring regenerated individuals even in difficult-to-culture varieties have been developed by introducing several genes (NPLs 1 and 2). However, use of transgenic technologies is essential in order to apply these methods, and there is still a concern about the approval in the practical stage. Also in other species, varieties and strains called difficult-to-culture varieties, which are difficult to reproduce individuals by tissue culture, are universally present, which is an obstacle to breeding.

Further, genome editing has been applied to breeding crops in recent years. However, practical use thereof is also hindered since the difference in ease of tissue culture depending on the crop species and variety greatly affects the production efficiency of genome edited products (NPL 3).

That is, if the difficulty that can occur during culture of plant cells or tissues and differs depending on the species and variety can be overcome, further progress in breeding using transgenic technologies and genome editing technologies would be enabled.

Since a fertilized plant egg is a cell whose direction of generation or differentiation has been determined, control of dedifferentiation and redifferentiation thereof necessary for normal cell or tissue culture is expected to be comparatively easy, and growth into a plant is considered to be possible by simply adjusting the culture conditions necessary for cells or tissues to be cultured. Further, since a fertilized egg cell is a single cell, it can be also expected that the genotype mosaicity of individuals can be avoided if a nucleic acid or protein for genome editing can be introduced into the cell in such a state.

For fertilized plant eggs, isolation methods, culture methods, and methods of introducing substances have been studied. For species such as maize (NPL 4), rice (NPL 5), wheat (NPLs 6 and 7), barley (NPLs 8 and 9), and tobacco (NPL 10), methods for isolating a fertilized egg cell are shown, and examples of producing a plant by continuing culture after the isolation are also known. Further, NPL 11 discloses a technique to obtain a fertilized egg immediately after fertilization by artificial insemination (in-vitro fertilization) of isolating a sperm cell and an egg cell from a maize plant and artificially fusing them, and a method to culture the fusion into a plant.

As examples of trying to introduce a substance into a fertilized plant egg, there have been reports of introducing DNA into maize, wheat, and barley by the microinjection method (NPLs 4, 7, and 9). The microinjection method enables gene introduction into a cell having a cell wall, and there is no particular need to remove the cell wall of a plant cell as an introduction target by a treatment with plant tissue-degrading enzymes or the like. However, there is a disadvantage that only one cell can be handled in one introduction operation, and it is not suitable for gene introduction experiments of middle to large scale using many plant cells. Further, since complicated operations under a microscope and skilled techniques are required for the microinjection, its' practical use has been difficult.

Further, a method for introducing a nucleic acid by applying the particle gun method to a fertilized egg contained in an ovary or ovule cut out without isolating the fertilized egg has been reported (PTL 1). However, in the particle gun method under the condition that many other tissues are present around the fertilized egg, the efficiency of introducing the nucleic acid into the fertilized egg itself is assumed to be poor, and there is no report on its practical application.

In recent years, techniques to introduce a substance such as a nucleic acid present around a fertilized egg by removing the cell wall of the fertilized egg by treatment with an appropriate enzyme or treating the fertilized egg immediately after fertilization having an undeveloped cell wall with polyethylene glycol have been reported (PTL 2, PTL 3, NPL 12, and NPL 13). A desired genome edited product has been obtained by introducing a genome editing tool composed of DNA or ribonucleoprotein into a fertilized rice egg using this method (NPL 13). Other than the aforementioned methods, methods for introducing a substance into a cell such as the peptide method (NPLs 14 and 15), the electroporation method, and the Agrobacterium method are known. However, studies to introduce a substance into a fertilized plant egg cell or a plant germ cell using such a method have not been reported.

That is, application of the PEG method is most reliable for introducing a substance such as a nucleic acid and a protein into a fertilized plant egg isolated or the like and growing the egg into a plant, to obtain a desired genome edited product, so far. However, the PEG method requires a high cell manipulation technique, and the number of fertilized eggs to be treated per unit time is limited. Therefore, although it is possible to introduce a substance into a plant cell using the PEG method, there has been a demand for establishment of a substance introduction method capable of genome editing on a fertilized plant egg or the like with higher efficiency.

Generally, the particle gun method is considered to be the most efficient method per unit time for introducing a substance into a cell. However, the redifferentiation efficiency per cell from the cell with the substance introduced after the particle gun treatment is not necessarily high. Further, the particle gun method is generally applied to cells and tissues present in the gas phase, and there have been few reports that the method is used for objects present in the liquid phase.

### CITATION LIST

### Patent Literature

PTL 1: WO1998001575A1
PTL 2: WO2017171092A1
PTL 3: WO2018143480A1

### Non Patent Literature

NPL 1: Lowe, K. et al., (2016), Plant Cell 28:1998-2015.
NPL 2: Mookkan, M., et al., (2017), Plant Cell Report 36:1477-1491.
NPL 3: Altpeter F. et al., (2016), Plant Cell 28:1510-1520.
NPL 4: Leduc, N. et al., (1996), Developmental Biology 177:190-203.
NPL 5: Zhang, J. et al., (1999), Plant Cell Reports 19: 128-132.
NPL 6: Kumlehn, J. et al., (1997), Plant Cell Reports 16: 663-667.
NPL 7: Ponya, Z. et al. Protoplasma (1999) 208:163-172.
NPL 8: Holm, P. B. et al., (1994), The Plant Cell 6: 531-543.
NPL 9: Holm, P. B. et al., (2000), Transgenic Research 9: 21-32.
NPL 10: Yuchi, H. E. et al., (2004), Chinese Science Bulletin 49:810-814.
NPL 11: Kranz E. and Loerz H., (1993), Plant Cell 5:739-746.
NPL 12: Koiso, N. et al., (2017), Plant Direct 1-10.
NPL 13: Toda, E. et al., (2019), Nature Plants 5:363-368.
NPL 14: Laksmanan, M. et. al., (2013), Biomacromolecules 14, 10-16.
NPL 15: Laksmanan, M. et al. (2015), Plant Biotechnology 32:39-45.
NPL 16: Daniell, H. et. al., (1990), Proc. Natl. Acad. Sci. USA 87:88-92.

### SUMMARY OF INVENTION

### Technical Problem

It is an object of the present invention to provide a method for introducing a substance into a plant cell, and a substance-introduced plant cell.

A fertilized plant egg cell is a cell that originally possesses the ability to grow into a plant and therefore is expected not to be affected by the difference in culture efficiency between species or varieties. Treatments such as transformation and genome editing can be performed on a wider variety of species or crops by introducing a substance into a fertilized egg cell that is a zygote, or an egg cell or a sperm cell that is a gamete as a target than in the current situation.

Before the present invention, there have been no reports of the particle gun method being applied to fertilized plant eggs that are normally maintained and cultured in the liquid phase. Up to now, there have been attempts to apply the particle gun method to cells cultured in a liquid, but these attempts are performed by taking out the cultured cells temporarily in the gas phase (NPL 16), and the particle gun method has not been applied to cells that are difficult to transfer from the solution to the gas phase, such as fertilized plant eggs and protoplast-like cells.

The inventors have found that a substance can be introduced into a plant cell present in the liquid phase such as fertilized egg cells using the particle gun method, thereby conceiving the present invention. The substance can be more easily introduced by adjusting various conditions for the particle gun method, particularly, the distance between the plant cell present in the liquid phase and the upper surface of the liquid phase.

### Solution to Problem

The present invention includes, though not limited to, the following embodiments.

### Embodiment 1

A method for introducing a substance into a plant cell, comprising introducing a substance into a plant cell present in a liquid phase using a particle gun method.

### Embodiment 2

The method according to embodiment 1, wherein the plant cell is any one of a plant germ cell, a fertilized plant egg cell, and a pre-embryonic cell group.

### Embodiment 3

The method according to embodiment 1 or 2, wherein the plant cell is a fertilized plant egg cell.

### Embodiment 4

The method according to embodiment 1 or 2, wherein the plant cell is a pre-embryonic cell group with the number of cells being 8 or less.

### Embodiment 5

The method according to embodiment 4, wherein the plant cell is a pre-embryonic cell group with the number of cells being 4 or less.

### Embodiment 6

The method according to embodiment 4 or 5, wherein the plant cell is a pre-embryonic cell group with the number of cells being 2 or less.

### Embodiment 7

The method according to any one of embodiments 1 to 3, wherein the plant cell is a fertilized plant egg cell having a cell wall.

### Embodiment 8

The method according to any one of embodiments 1 to 7, wherein the distance between the plant cell and the upper surface of the liquid phase is more than 0 μm.

### Embodiment 9

The method according to any one of embodiments 1 to 7, wherein the distance between the plant cell and the upper surface of the liquid phase is 50 μm or more.

### Embodiment 10

The method according to any one of embodiments 1 to 9, wherein the distance between the plant cell and the upper surface of the liquid phase is 3000 μm or less.

### Embodiment 11

The method according to any one of embodiments 1 to 9, wherein the distance between the plant cell and the upper surface of the liquid phase is 2000 μm or less.

### Embodiment 12

The method according to any one of embodiments 1 to 11, wherein the substance to be introduced is selected from the group consisting of nucleic acids, proteins, peptides, polysaccharides, lipids, and cell organelles.

### Embodiment 13

The method according to any one of embodiments 1 to 12, wherein the liquid phase is a solution in a medium containing a gelling agent.

### Embodiment 14

The method according to any one of embodiments 1 to 13, wherein the plant cell is a cell of a monocotyledonous plant.

### Embodiment 15

The method according to embodiment 14, wherein the plant cell is a cell of a gramineous plant.

### Embodiment 16

The method according to embodiment 15, wherein the plant cell is a cell of a plant selected from the group consisting of maize, wheat, barley, rice, and sorghum.

### Embodiment 17

The method according to any one of embodiments 1 to 16, comprising the steps of:

step (1) of obtaining a plant cell that is any one of a plant germ cell, a fertilized plant egg cell, and a pre-embryonic cell group;

step (2) of culturing the plant cell obtained in step (1) in the liquid phase, wherein the distance is adjusted between the plant cell obtained in step (1) and the upper surface of the liquid phase; and

step (3) of introducing the substance into the plant cell present in the liquid phase using the particle gun method.

### Embodiment 18

A substance-introduced plant cell obtained by the method according to any one of embodiments 1 to 17.

### Embodiment 19

A substance-introduced plant comprising the plant cell of embodiment 18.

### Advantageous Effects of Invention

Conventionally, the microinjection method and the polyethylene glycol method have been known as methods for introducing a substance into a fertilized plant egg and obtaining a plant from the fertilized egg. Since both of these methods require a high level of cell manipulation technique and a long time for the introduction operation, there has been a demand for the development of a more efficient method for introducing a substance into a fertilized egg cell. According to the present invention that applies a method of attaching a substance to be introduced to gold particles and emitting the gold particles using gas pressure or the like, that is, a so-called particle gun method to a fertilized plant egg, the efficiency of introducing a substance into a fertilized plant egg has been improved as compared with the conventional art, and the number of fertilized eggs that can be treated by one worker per unit time has been significantly increased. Further, a method for culturing the fertilized plant egg is combined therewith, thereby inducing division from the substance-introduced cell, to enable genome editing and transformation.

### BRIEF DESCRIPTION OF DRAWINGS

The patent or application file contains at least one color drawing. Copies of this patent or patent application publication with color drawing will be provided by the USPTO upon request and payment of the necessary fee.

FIG. 1 includes photographs showing morphological changes after particle gun treatment due to the difference in distance from a fertilized maize egg cell to the upper surface of the liquid phase (water depth). Specifically, they are inverted micrographs of a fertilized egg treated with a particle gun 1 week after culture. A: Water depth: 1.0 mm, B: Water depth: 1.25 mm, C: Water depth: 1.5 mm, D: Water depth: 1.75 mm, E: Water depth: 2.0 mm, F: Untreated section (water depth: 1.25 mm)

FIG. 2 is a fluorescence micrograph of rice cultured cells (Oc cells) 3 days after introduction of a GFP plasmid into Oc cells in a liquid medium treated with a particle gun.

FIG. 3 is a fluorescence micrograph of fertilized maize eggs 3 days after introduction of a GFP plasmid into the fertilized eggs during culture in a liquid medium containing a gel.

FIG. 4 is a fluorescence micrograph of fertilized maize eggs 3 days after introduction of a GFP plasmid into the fertilized eggs during culture in a liquid medium not containing a gel.

FIG. 5 is a fluorescence micrograph of callus derived from fertilized eggs 1 week after introduction of GFP plasmid into the fertilized eggs during culture in a liquid medium.

### DESCRIPTION OF EMBODIMENTS

I. Method for Introducing Substance into Plant Cell

The present invention relates to a method for introducing a substance into a plant. The method of the present invention comprises introducing a substance into a plant cell present in the liquid phase using the particle gun method.

The method of the present invention comprises introducing a substance after isolation into a plant germ cell, a fertilized plant egg cell, or a pre-embryonic cell group with the number of cells that have started to divide being 8 or less, which are present in the liquid phase.

According to one embodiment, the method for introducing a substance into a plant comprises the steps of:

(1) obtaining a plant cell that is any one of a plant germ cell, a fertilized plant egg cell, and a pre-embryonic cell group,

(2) culturing the plant cell obtained in step (1) in the liquid phase, wherein the distance is adjusted between the plant cell obtained in step (1) and the upper surface of the liquid phase, and

(3) introducing the substance into the plant cell present in the liquid phase using the particle gun method.

Plant

The types of the plant are not specifically limited. Any one of dicotyledonous plants and monocotyledonous plants may be employed, and preferably, monocotyledonous plants are employed. Further preferably, gramineous plants, more preferably, maize, wheat, barley, rice, sorghum, rye, or the like, and most preferably, maize, wheat, rice, or sorghum is employed.

The method of the present invention can be particularly used for, though not limited to, “difficult-to-culture” plants or varieties, since it targets fertilized plant eggs, fertilized plant egg cells, or pre-embryonic cell groups. The term “difficult-to-culture” means that culture is difficult, specifically, culture of a cell isolated from a plant is difficult, callus formation by a treatment such as dedifferentiation is difficult, or redifferentiation from callus into a plant is difficult, for example.

Generally, culture of monocotyledonous plants is more difficult than that of dicotyledonous plants, but the “difficult-to-culture” plants, for example, include soybeans, common beans, capsicums, and the like. The difficult-to-culture varieties mean varieties culture of which is more difficult than that of general research varieties (such as maize A188) of the same species. Examples thereof include maize B73, maize elite varieties derived from B73, wheat elite varieties (such as AC Barrie and TAM), barley varieties other than GoldenPromise and Igri, and sorghum varieties other than 296B, C401, SA281, P898012, Pioneer 8505, and Tx430.

Plant Cell

The method of the present invention comprises introducing a substance into a plant cell.

The plant cell used in the method of the present invention is not specifically limited, as long as it is a plant cell that can be cultured (including storage) in the liquid phase. According to the method of the present invention, the substance can be introduced using the particle gun method in the state where the plant cell is present in the liquid phase.

Without limitation, the plant cell is any one of a plant germ cell, a fertilized plant egg cell, and a pre-embryonic cell group. Such cells are generally cultured in the liquid phase, and thus it has been considered before the present invention that the particle gun method cannot be used therefor.

The germ cell is a cell playing a role in transmitting genetic information to the next generation through reproduction and is a cell other than somatic cells among cells constituting a multicellular organism. In this description, a “germ cell” includes an egg cell (referred to also as “egg” or “ovum”) and a sperm cell (referred to also as “sperm”).

The plant germ cell is preferably an egg cell or a sperm cell isolated from a stamen or a pistil. In this description, an “egg cell” means a female gamete formed by meiosis of the embryo sac mother cell in the pistil. Although the method for isolating an egg cell is not limited, the ovary is cut in a solution with an appropriate osmotic pressure, and then an egg cell exposed on the cut surface can be isolated using a glass capillary under a microscope, for example. In this description, a “sperm cell” means a male gamete formed by meiosis of a pollen mother cell in the anther of a stamen. Although the method for isolating a sperm cell is not limited, upon immersion of pollen collected from the anther in a solution with an appropriate osmotic pressure, the contents of the pollen containing a sperm cell are released from the pollen into the solution after a lapse of several minutes, and then a sperm cell can be isolated using a glass capillary under a microscope, for example.

In this description, a “fertilized egg cell” means a cell in which a sperm cell and an egg cell are fused. Without limitation, the fertilized egg cell is preferably isolated from a tissue of a plant containing the embryo sac but may be obtained by fertilizing an egg cell and a sperm cell isolated under a microscope.

The plant cell may be a fertilized plant egg cell having a cell wall or may be a cell with incomplete cell wall formation. Since the plant cell has a cell wall, a tissue and a cell are generally treated with plant tissue-degrading enzymes such as cellulase, pectinase, protease, and hemicellulase particularly in the PEG method or the electroporation method, to dissolve the cell wall, thereby protoplastizing the cell. Since the method of the present invention uses the particle gun method, a substance can be introduced even into a fertilized plant egg cell having a cell wall. Without limitation, the plant cell is a fertilized plant egg cell having a cell wall in one embodiment.

The “cell with incomplete cell wall formation” is not a cell like a plant cell, which is almost entirely surrounded by a cell wall except plasmodesmata connection but a cell not surrounded by a cell wall partially or entirely. The plant cell with incomplete cell wall formation includes gametes for sexual reproduction including an egg cell and a sperm cell. Alternatively, it includes a fertilized egg cell which is formed by fertilization and in which cell wall formation has not started or cell wall formation has started but has not been completed yet in an incomplete state. Cell wall formation in a plant germ cell can be checked, for example, by a known method such as cellulose staining by Calcofluor-white and aniline blue staining.

The plant cell may be a pre-embryonic cell group, that is, cells in a pre-embryonic stage. In this description, the “pre-embryonic stage” refers to the period until a fertilized egg that has started to divide reaches the 8-cell stage. Generally, a fertilized egg is in a stage after fertilization or fusion of an egg cell and a sperm cell, but the fertilized egg may then start to divide in a short time to be multicellular. When a substance is introduced into a pre-embryonic cell group that has become multicellular from a fertilized egg according to the present invention, a mosaic transformant or a chimeric genome edited product may be formed. However, these can eliminate the mosaism in the progeny, and the same effect as when the process of the present invention is applied to the fertilized egg can be expected. According to one embodiment of the present invention, the plant cell into which the substance is introduced is a pre-embryonic cell group with the number of cells being 8 or less, a pre-embryonic cell group with the number of cells being 4 or less, or a pre-embryonic cell group with the number of cells being 2 or less. The case where the number of plant cells is 2 to 8 corresponds to the pre-embryonic cell group. The number of plant cells can be examined, for example, by placing the cells under a microscope and counting them visually. Further, it is also possible to grasp the number of cells based on the number of days after the start of culture by grasping the development process of the fertilized egg in advance.

Method for Obtaining Fertilized Egg Cell

According to one embodiment of the present invention, an egg cell and a sperm cell of a plant may be fused in vitro to produce a fertilized egg cell. That is, an egg cell and a sperm cell can be first isolated from a plant to produce a fertilized egg cell in vitro by a known method such as electrofusion (also referred to as gamete fusion).

Electrofusion is a method of fusing two or more cells in vitro by electric stimulation. Specifically in this description, a fertilized egg cell can be produced by applying an electric pulse to an egg cell and a sperm cell isolated in a solution with an appropriate osmotic pressure and thereby fusing the cells.

In the case of fusing the cells by electrofusion, conditions such as voltage and distance between electrodes can be appropriately determined by those skilled in the art corresponding to the type of the plant or the sizes of the cells. For example, the conditions disclosed in Japanese Patent Laid-Open No. 2016-63785 (PTL 1) can be used.

When one fusion cell (a fertilized egg cell) is produced by electrofusion of a sperm cell and an egg cell, the lower limit of the DC voltage is preferably 10 kV or more, more preferably 11 kV or more, further preferably 12 kV or more. Further, the upper limit is preferably 17 kV or less, more preferably 16 kV or less, further preferably 15 kV or less. The upper limit and the lower limit can be appropriately selected by those skilled in the art.

Further, the lower limit of the distance between electrodes is preferably 1.5 times or more, more preferably 2 times or more, further preferably 2.5 times or more, most preferably 3 times or more of the sum of the diameters of the egg cell and the sperm cell to be fused. Further, the upper limit is preferably 6 times or less, more preferably 5 times or less, further preferably 4 times or less. The upper limit and the lower limit can be appropriately selected by those skilled in the art. Examples of the method for measuring the diameter of a cell include a method of measuring the diameter using a micrometer eyepiece mounted on a microscope, and a method of importing an image captured with a microscope into a computer and measuring the diameter using an image analysis software.

Further, the distance between electrodes can be appropriately selected by those skilled in the art. For example, the lower limit is preferably 80 μm or more, more preferably 90 μm or more, further preferably 100 μm or more. Further, the upper limit is preferably 240 μm or less, more preferably 220 μm or less, further preferably 200 μm or less. The upper limit and the lower limit can be appropriately selected by those skilled in the art.

The osmotic pressure of the solution used when one fusion cell is produced by electrofusion of a sperm cell and an egg cell can be appropriately selected corresponding to the type of the plant to be used. For example, in the case of rice, the lower limit is preferably 380 mosmol/kg H₂O or more, more preferably 390 mosmol/kg H₂O or more, further preferably 400 mosmol/kg H₂O or more. Further, the upper limit is preferably 470 mosmol/kg H₂O or less, more preferably 460 mosmol/kg H₂O or less, further preferably 450 mosmol/kg H₂O or less. In the case of maize, the lower limit is preferably 600 mosmol/kg H₂O or more, further preferably 630 mosmol/kg H₂O or more. Further, the upper limit is preferably 700 mosmol/kg H₂O or less, further preferably 680 mosmol/kg H₂O or less. The upper limit and the lower limit can be appropriately selected by those skilled in the art.

Alternatively, other known cell fusion methods such as the calcium fusion method and the PEG fusion method may be used for cell fusion of an egg cell and a sperm cell. The “calcium fusion method” utilizes a property of cell membranes that fusion of cell membranes tends to occur depending on the calcium concentration. The “PEG fusion method” utilizes cell fusion by treating a cell with polyethylene glycol (PEG) to bind cell membranes, followed by removal of PEG.

Alternatively, a fertilized egg cell may be produced in a plant by natural fertilization to obtain the fertilized egg cell produced from the plant. The method for obtaining a fertilized egg cell using natural fertilization is, for example, a method of exposing the stigma, attaching pollen for pollination, and thereafter isolating a fertilized egg cell from a tissue containing the embryo sac. The fertilized egg cell can be isolated from the plant by extracting the ovary immediately after fertilization from the plant after pollination, cutting the ovary in a solution with an appropriate osmotic pressure, and isolating the fertilized egg cell exposed on the cut surface under a microscope using a glass capillary or the like. Alternatively, the fertilized egg cell can be released to be isolated, for example, by dissecting a tissue such as nucellus using a glass needle under a microscope, after treatment of the ovary or ovules with an enzyme solution for a certain time. In this description, the natural fertilization may be artificial mating in which pollen is artificially attached to the stigma or may be natural mating.

According to one embodiment, the method for introducing a substance into a plant cell may comprise first introducing a substance into either or both of an egg cell and a sperm cell, and then producing a fertilized egg. Without limitation, the method comprises:

(1) introducing a substance into either or both of an egg cell and a sperm cell of a plant using the particle gun method, and

(2) fusing the egg cell and the sperm cell that have undergone step (1) to produce a fertilized egg cell.

Substance to be Introduced into Plant Cell

In the present invention, the substance introduced into the plant is a substance with a size and properties which can be supplied into a cell that is a target. The substance may be naturally present or artificially produced. Examples thereof include various biomolecules and compounds. Examples of the biomolecules include nucleic acids, proteins, peptides, polysaccharides, lipids, and cell organelles. According to one embodiment, the substance is selected from the group consisting of nucleic acids, proteins, and peptides.

The nucleic acids are not specifically limited and may be RNA, DNA, and a conjugate or mixture of the two. Preferably, the nucleic acids are circular DNA like vectors, linear DNA, circular RNA, or linear RNA. The nucleic acid may be double-stranded, single-stranded, or triple-stranded or more. It also includes an embodiment in which most are double-stranded and only the ends are partially single-stranded. The size of the nucleic acid is not specifically limited, as long as the size allows introduction into a plant cell by the particle gun method. Without limitation, the length is preferably 100 kbp or less, more preferably 40 kbp or less, further preferably 20 kbp or less.

Proteins for genome editing such as nucleases including Cas9 nuclease, modifying enzymes, and antibodies can be introduced, for example. Without limitation, the size of the proteins is a molecular weight of preferably 300 kDa or less, more preferably 200 kDa or less, further preferably 150 kDa or less.

Peptides generally refer to molecules in which various amino acids are linked in a fixed order by amide bonds (also referred to as “peptide bonds”) and generally have a shorter length than proteins. Preferably, the length is 100 a.a. or less, more preferably 50 a.a. or less.

“Polysaccharides” generally refer to materials in which two or more monosaccharide molecules are polymerized by glycosidic bonds. For example, they exhibit properties different from those of monosaccharides which are constituent units, like starch. Examples thereof include starch (amylose and amylopeptin), glycogen, cellulose, chitin, agarose, carrageenan, heparin, hyaluronic acid, pectin, xyloglycan, and glucomannan.

“Lipids” generally refer to substances that are isolated from organisms and are insoluble in water. They are defined by the solubility, not by specific chemical or structural properties. In the biochemical definition, they are “in-vivo or biologically-derived molecules having long-chain fatty acids or hydrocarbon chains”. Examples thereof include: (i) simple lipids (such as acyl glycerol and ceramide) formed by ester bonding of only an alcohol and a fatty acid, (ii) complex lipids (such as phospholipid, sugar lipid, and lipoprotein) containing a phosphate or sugar in the molecule and generally having a skeleton of sphingosine or glycerin, and (iii) derived lipids (such as fatty acid, tempenoid, steroid, and carotenoid) that are hydrophobic compounds derived from simple lipids or complex lipids by hydrolysis.

“Cell organelles” generally refer to structures with specifically differentiated morphologies or functions inside cells and may be called intracellular organs or organelles. According to one embodiment, examples thereof include structures surrounded by biomembranes such as nuclei, endoplasmic reticula, the Golgi apparatus, endosomes, lysosomes, mitochondria, chloroplasts, and peroxisomes. Examples further include cell skeletons and structures composed of supercomplexes of non-membrane proteins such as centrosomes, flagella, and cilia. Examples can further include nucleoli and ribosomes.

The “substance” can also include metal ions, compounds, and the like other than biomolecules. Two or more types of substances such as nucleic acids, proteins, peptides, polysaccharides, lipids, metal ions, and compounds may be introduced in combination. For example, nucleic acids may be two or more types of DNAs or RNAs, or a combination of DNA and RNA. Different types of substances such as a nucleic acid and a protein may be introduced simultaneously or as a complex.

Further, the substances may be supplied while being carried by or contained in a carrier for introduction depending on the properties of substances to be introduced. Here, the carrier is a medium (vehicle) for supplying foreign substances into a cell. Examples thereof include liposomes, particles or whiskers composed of metals (such as gold and tungsten) or inorganic substances (such as silicon compounds), alginate beads, and viral substances (such as coating proteins). Further, substances that promote substance introduction into a cell may be supplied simultaneously. Examples thereof include proteins such as fibronectin, peptides, chelate compounds (such as EDTA), and inorganic microstructures (such as nanostructured silica).

Adjustment of Distance Between Plant Cell into which Substance is Introduced and Liquid Phase (Water Depth)

The method for introducing a substance into a plant cell of the present invention comprises introducing a substance into a plant cell present in the liquid phase using the particle gun method.

The distance between the plant cell into which the substance is introduced and the upper surface of the liquid phase (which may be referred to as “liquid surface” in this description) is adjusted at any time immediately after isolation of the fertilized egg and before introduction of the substance. The distance between the fertilized egg and the liquid surface can be adjusted by adjusting the amount of the medium to be put into a Petri dish and millicells to be provided in the Petri dish. The lower limit of the distance between the plant cell into which the substance is introduced and the upper surface of the liquid phase needs only to be greater than 0 μm, that is, there is no limitation as long as the cell is completely inside the liquid phase. When the cell is exposed from the liquid surface, osmotic shock or the like may possibly occur. The distance between the plant cell and the upper surface of the liquid phase is preferably 50 μm or more, or 100 μm or more, further preferably 500 μm. Meanwhile, the upper limit is a distance that allows gold particles to reach the fertilized egg. The upper limit is preferably 5000 μm or 4000 μm, further preferably 3000 μm, more preferably 2000 μm or less, 1750 μm or less, 1500 μm or less, or 1250 μm or less. Those skilled in the art can appropriately adjust the distance corresponding to equipment parameters based on such a value. For example, in the case of using a plastic Petri dish with a diameter of 3.5 mm, a water level of about 1000 μm can be raised by adding 1 ml of a medium.

The liquid phase in which the plant cell is present during introduction of the substance using the particle gun method is not specifically limited. Depending on the type of the plant from which the plant cell is derived and the state of the cell (such as a fertilized egg cell, a sperm cell, and an egg cell), etc., a medium suitable for culturing the plant cell can be appropriately used, as it is, for the substance introduction using the particle gun method. According to one embodiment, the “medium” in this description is a medium for culturing the plant cell and may be a medium containing carbon sources, nitrogen sources, salts, and the like, which are necessary for culturing the plant cell (such as, though not limited to, MS medium, B5 medium, N6 medium, or ZMS medium). Alternatively, the “medium” may be a medium into which the plant cell isolated is moved before transplantation into a culture medium and which contains a substance for adjusting the osmotic pressure, such as mannitol, and the like. The substance may be introduced into the plant cell in the medium into which the plant cell isolated is moved before transplantation into a culture medium, and then the cell may be transferred into the culture medium.

According to one embodiment, the liquid phase in which the plant cell is present may be a solution in a medium containing a gelling agent. That is, the medium for culturing the plant cell into which the substance is introduced may contain a gelling agent known in the technical field. Without limitation, examples of the gelling agent include agarose, agar, gellan gum, gelrite, alginic acid, gelatin, and phytagel. The gelling agent contained in the medium may be of one type or two or more types (such as two types, three types, four types, or more types).

The concentration of the gelling agent that can be contained in the medium is appropriately selected depending on the type of the gelling agent. Preferably, the concentration is selected such that a composition containing at least one gelling agent is not completely solid, but has a semi-solid state containing a liquid state or a solution portion. For example, in the case of agarose, 0.05 to 2.0% is preferable, 0.1 to 1.0% is more preferable, 0.2 to 0.5% is further preferable, and 0.3 to 0.4% is most preferable.

In this description, the “liquid state” means that the composition (such as a solution) containing the gelling agent is in liquid form and is in a state of taking a fluid without a fixed shape. The “semi-solid state” means a state having both properties of a liquid and a solid and is a so-called gel state characterized by being viscous and freely deformable.

Substance Introduction

The method for introducing a substance into a plant of the present invention uses the particle gun method. The “particle gun method” is a method of attaching the substance to be introduced to gold particles and emitting the gold particles toward a target using gas pressure or the like.

Although not limited, a PDS-1000/He(TM) system (manufactured by Bio-Rad Laboratories, Inc.) can be used as a device for emitting the gold particles. Using a suspension of the gold particles adjusted to a concentration of 60 mg/l in 50% glycerol, the amount of the gold particle suspension to be used is adjusted corresponding to the number of times of treatment. The size of the gold particles is not limited but is preferably a diameter of 0.6 μm, 1.0 μm, or 1.6 μm. Hereinafter, the case of performing the treatment 5 times will be shown as an example.

The amount of reagents in each step, the number of times of treatment, and the like are not limited. 50 μl of the gold particle suspension is put into a tube. 50 μl of 2.5 M CaCl₂) and 20 μl of 0.1 M spermidine are put therein. Thereafter, the mixture is stirred with a shaker for 3 minutes, and then treated with a centrifuge at 10000 rpm for 3 minutes, to remove the supernatant. Then, it is washed once with 70% ethanol and twice with 100% ethanol. 150 μl of ethanol is added in each time of washing. After treatment with a stirrer (Voltex-GENIE2, Scientific Industries Inc.), treatment is performed with an ultrasonic cleaner until the gold particles are uniformly diffused in the solution, and the supernatant is removed after centrifugation. After the completion of washing 3 times, 100% ethanol is added thereto, to obtain a gold particle suspension to be applied to microcarrier paper.

The microcarrier paper is immersed in 70% ethanol to remove impurities and then spread on a paper towel to evaporate the ethanol. Next, the microcarrier paper is set in a predetermined holder attached to PDS-1000/He (Bio-Rad Laboratories, Inc.). 10 μl each of the gold particle suspension is added dropwise at the center of the microcarrier paper set in the holder and dried at room temperature.

Thereafter, the gold particles can be injected into an object, for example, using a 650 PSI rupture disc with a pressure set to 5.9 μPa.

Callus Formation or Embryo-Like Structure (Cell Mass) Formation and Redifferentiation

The method for introducing a substance into a plant of the present invention may further comprise: conducting callus formation or embryo-like structure formation of the plant cell into which the substance has been introduced, after the step of introducing the substance; and redifferentiating the callused or embryonated tissue in a redifferentiation medium.

The callus formation or embryo-like structure formation step and the redifferentiation step are not specifically limited, and known methods for regenerating a plant from a fertilized egg cell can be used.

In the callus formation or embryo-like structure formation step, the substance-introduced fertilized egg cell obtained is cultured to form the embryo-like structure or callus. The step of inducing division of a fertilized egg cell and allowing the cell to grow so as to form the callus or embryo-like structure is not specifically limited, since the optimal conditions differ depending on plants, but is preferably the nurse culture method with feeder cells added. For example, the procedure can be as follows.

Pretreatment: The substance-introduced fertilized egg cell may be put into a mannitol droplet (600 mosmol/kg H₂O) for washing, so as to be sterilized.

Culture in liquid medium: The substance-introduced fertilized egg cell is transferred into a medium and cultured by gentle shaking, but the culture can be continued as it is in a culture liquid at the time of the substance introduction. The shaking speed is preferably 30 to 50 rpm, more preferably 35 to 45 rpm. The culture temperature is preferably 24 to 28° C., more preferably 25 to 27° C. The culture is preferably performed in the dark. At this time, feeder cells are preferably added to the medium to perform co-culture (nurse culture method). The culture period is preferably 4 to 14 days, more preferably 5 to 10 days.

Medium: Liquid MS medium (T. Murashige et al., Physiol. Plant., 15, 473 (1962)), B5 medium (O. L. Gamborg et al, Experimental Cell Research, 50, 151-158 (1968)), or N6 medium (Chu et al., Sci. Sinica, 18, 659-668 (1975)), and the like, to which auxins such as 2,4-dichlorophenoxyacetic acid and naphthaleneacetic acid are added.

Auxins such as indole-3-acetic acid, 2,4-D, and dicamba are preferably added to the medium. The concentration of auxins to be added is, for example, 0.1 to 3.0 mg/L, preferably 0.1 to 0.3 mg/L, more preferably 0.15 to 0.25 mg/L. Depending on the variety, cytokinins such as zeatin may be added. The concentration is, for example, 0.5 to 10.0 mg/L but is preferably 2.0 to 8.0 mg/L.

Feeder cells: Any known feeder cells can be used. Examples thereof include a rice cell suspension culture (Line Oc, manufactured by Riken BioResource Research Center), a maize nurse cell (Mol et al., 1993), and a non-morphogenic cell suspension (Kranz et al., 1991).

By this step, a spherical embryo-like structure with a diameter of about 50 to 200 μm is formed 4 to 14 days after the start of culture.

The redifferentiation step also can be carried out according to a known redifferentiation step. For example, it can be performed, as follows.

Culture: A spherical embryo-like structure is transferred to a medium not containing feeder cells, followed by further culture for about 10 to 14 days. Thereafter, the embryo-like structure is cultured in an arbitrary medium not containing auxins such as MS medium to form a plant. Cytokinins such as zeatin may be added at a concentration of 0.5 to 10.0 mg/L in order to promote shoot regeneration. The concentration is further preferably 1.0 to 5.0 mg/L. At this time, the culture is further preferably performed under irradiation with light, and the irradiation is preferably at 50 to 180 μmol/m²·sec, more preferably 70 to 150 μmol/m²·sec, for example.

Medium: Solid media such as MS medium, B5 medium, and N6 medium, using agarose, agar, gellan gum, gelrite, or the like are mentioned, for example.

II. Substance-Introduced Plant Cell and Substance-Introduced Plant

The present invention further includes a substance-introduced plant cell obtained by the method of the present invention and a substance-introduced plant containing such a plant cell. Before the present invention, it has been difficult or impossible to obtain substance-introduced plants, particularly, of “difficult-to-culture” plants and varieties. The present invention enables a substance-introduced plant of such plants and varieties to be efficiently obtained by a simple method.

Further, the substance-introduced plant obtained by the method of the present invention includes not only plants with nucleic acids such as plasmids and gene sequence fragments, proteins or peptides for genome editing or the like being introduced and held within the plants, but also transformed plants obtained by introducing substances, particularly, genes, genome-edited plants by introduction of substances associated with genome editing such as Cas9 and guide RNA, and their progenies and clones.

### EXAMPLES

Hereinafter, the present invention will be described in detail based on examples, but the present invention is not limited to these examples. Those skilled in the art can easily modify and change the present invention based on the disclosure of this description, and such modifications and changes are included in the technical scope of the present invention.

### Example 1 Preparation of Medium Used for Culturing Fertilized Egg Cells

0.2 mL of a medium for fertilized cells was prepared. The medium for fertilized cells was ZMS medium (Kranz, 1993). The difference from the MS medium was that 165 mg/L NH₄NO₃, and 1.0 mg/L nicotinic acid, 10.0 mg/L thiamine.H₂O, 1 mg/L pyridoxine.HCl, 750 mg/L glutamine, 150 mg/L proline, 100 mg/L asparagine, and 100 mg/L myoinositol as organic substances were added. 2 mg/L NAA or 2,4-D was added as a plant hormone, and glucose was further added thereto, to adjust the osmotic pressure to 600 mosmol/kg H₂O. The pH was 5.7.

The medium for fertilized cells prepared was put into a Millicell CM insert (manufactured by Merck KGaA) with a diameter of 12 mm, and it was put into a 3.5 cm plastic Petri dish containing 2 mL of a medium. Further, 40 to 60 μL of a rice cell suspension culture (Line Oc, manufactured by Riken BioResource Research Center) was added to the Petri dish as a feeder cell, to prepare a medium for culturing fertilized egg cells.

### Example 2 Isolation of Fertilized Maize Egg Cell

The tip portion of an ear was covered with a cap or the like so that pollen and the like would not adhere to the ear of maize (A188) grown in a greenhouse for pollination, and the ear was left until the mating period. The ear in the mating period was collected, the foreskin was removed in the laboratory to expose the ovary in the ear. The pistil (silk) emerging from each ovary with a length adjusted to about 12 cm with scissors was pollinated with pollen collected from the tassel of maize. The ear with the pollinated pistil and ovary was divided into two in the longitudinal direction and placed on an agar medium. The mating time was about 9:00 a.m. The ear after mating was stored in a temperature-controlled room at 25° C. and was stored under the same temperature conditions until the fertilized egg was extracted at 5° C. after a lapse of 12 hours.

A nucellus slice containing the embryo sac was extracted from the ovary of the ear of the day after mating and was put into 1 mL of a 10% mannitol solution (650 mosmol/kg H₂O) in a 3.5 cm plastic Petri dish. 0.5 mL of an enzyme mixture was put into the 3.5 cm plastic Petri dish, to give 1.5 mL of an enzyme solution, followed by standing at 32° C. for 30 to 60 minutes. The enzyme used was as follows. Cellulase (manufactured by Worthington Biochemical Corporation), Macerozyme R10 (manufactured by Yakult Honsha Co., Ltd.; galacturonase activity: 0.5 Unit/mg), Pectolyase Y23 (manufactured by Morishin Pharmaceutical Co., Ltd.; pectin lyase activity: 1 Unit/mg), or Sumiteam AP2 (manufactured by SHINNIHON CHEMICALS Corporation; galacturonase activity: 12.4 Unit/mg) dissolved in a 10% mannitol solution (650 mosmol/kg H₂O) to each concentration shown in Table 1 was adjusted to a pH of 7, followed by filter sterilization.

TABLE 1
Composition of enzyme solution

| Enzyme name | Concentration (%) |
|---|---|
| Cellulase | 1 |
| Macerozyme | 0.3 |
| Pectolyase | 0.05 |

A fertilized egg cell was isolated using two glass needles. The nucellus slice was fixed with one of the glass needles so as not to move, and tissues in the area in which the fertilized egg cell was estimated to be present were scraped out with the other glass needle, thereby isolating the fertilized egg cell. When fertilization is carried out, one of the two auxiliary cells invaded by the pollen tube is denatured and turned dark brown. The area was estimated using it as a mark. The fertilized egg cell isolated was washed with a 600 mosmol/kg H₂O mannitol solution using a micropipette and moved into a millicell provided in a liquid medium. The composition of the liquid medium was adjusted as described in Example 1.

The fertilized egg cell isolated was cultured in the dark at 26° C. and was tested for substance introduction on the next day.

### Example 3 Preparation of Gold Particle Suspension Used in Particle Gun and Introduction of Nucleic Acid by Particle Gun Method

A suspension of gold particles prepared at a concentration of 60 mg/l in 50% glycerol was used, and the amount of the gold particle suspension to be used was adjusted corresponding to the number of times of treatment. 1.0 μm gold particles were used. Hereinafter, the case of performing the treatment 5 times will be shown as an example. 50 μl of the gold particle suspension was put into a tube. 50 μl of 2.5 M CaCl₂, 20 μl of 0.1 M spermidine, and 4 μg of a plasmid containing GFP (green fluorescence protein) gene were put therein. Thereafter, the mixture was stirred with a shaker for 3 minutes and then treated with a centrifuge at 10000 rpm for 5 minutes, to remove the supernatant. Then, it was washed once with 70% ethanol and twice with 100% ethanol. 150 μl of ethanol was added in each time of washing. Specifically, washing was performed by treating the solution with ethanol added with a stirrer (Voltex-GENIE2, Scientific Industries Inc.) and then treating it with an ultrasonic cleaner until the gold particles were uniformly diffused in the solution, followed by centrifugation, to remove the supernatant. After the completion of washing 3 times, 100% ethanol was added thereto, to obtain a gold particle suspension to be applied to microcarrier paper.

The microcarrier paper was immersed in 70% ethanol to remove impurities and then spread on a paper towel to evaporate the ethanol. Next, the microcarrier paper was set in a predetermined holder attached to PDS-1000/He (Bio-Rad Laboratories, Inc.). 10 μl each of the gold particle suspension was added dropwise at the center of the microcarrier paper set in the holder and dried at room temperature.

In Example 4 and later, which will be described below, the nucleic acid was introduced by the particle gun method under the following conditions, unless otherwise specified.

The gold particles were injected into an object using a PDS-1000/He(TM) system, manufactured by Bio-Rad Laboratories, Inc., and a 650 PSI rupture disc (manufactured by Bio-Rad Laboratories, Inc.) with a pressure set to 5.9 μPa. The conditions used for injecting the gold particles were as follows. The number of times of injecting was once, and the distance from the stopping screen to the sample was 9 cm. The gold particles were injected while the lid of the Petri dish in which the culture had been started was open, and the fertilized egg cells were put in the millicell.

The fertilized egg cells used for introducing the nucleic acid by the particle method in Example 4 and later were obtained by culturing the fertilized egg cells isolated in the dark at 26° C. overnight (for about 24 hours) and were a pre-embryonic cell group with the number of cells being about 1 to 2.

### Example 4 Morphological Changes of Cells Due to Difference in Distance Between Maize Cultured Cells and Liquid Surface

In this example, the morphological changes after the particle gun treatment due to the difference in distance from the maize cells to the liquid surface (water depth) were investigated.

The maize cells used were obtained by culturing the fertilized egg cells isolated in Example 2 in the dark at 26° C. overnight (for about 24 hours) and were a pre-embryonic cell group with the number of cells being about 1 to 2. The nucleic acid was introduced by the particle gun method according to the method of Example 3, unless otherwise specified.

The distance between the cell and the surface of the culture liquid was adjusted by adjusting the amount of the medium to be put into the culture vessel. The medium was the same as that used for culturing the plant cell. After adjusting the distance, the gold particles were injected into the cell serving as a target using a particle gun device. The distance from the stopping screen to the cell was about 9 cm. About 5 fertilized eggs were put in each millicell provided in a Petri dish with a diameter of 3.5 cm. 2.0 ml, 2.25 ml, 2.5 ml, 2.75 ml, and 3.0 ml of the liquid medium were put into respective Petri dishes to the same level as the liquid surface in each millicell.

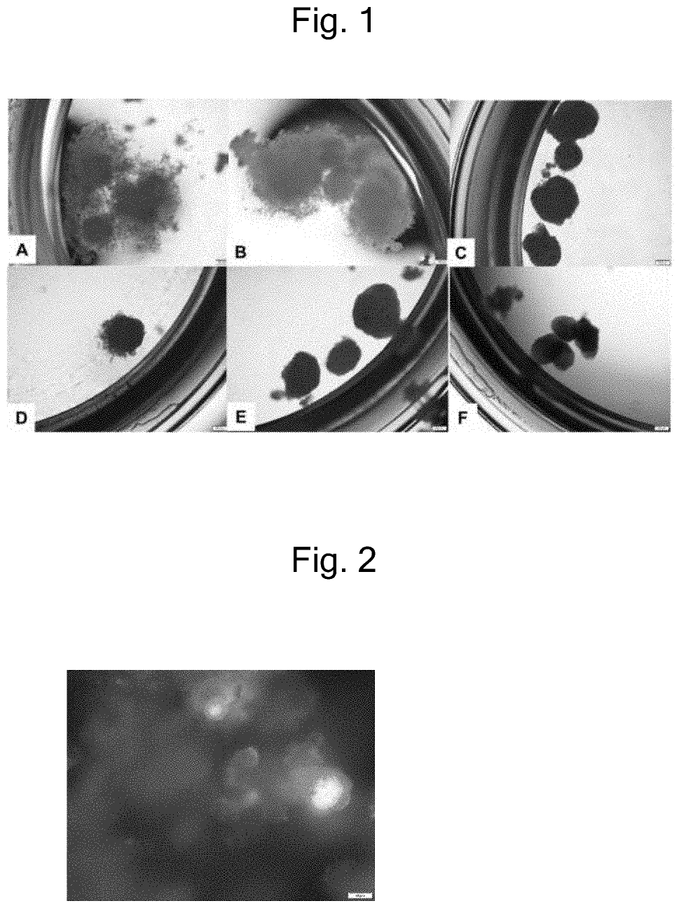

FIG. 1 and Table 2 show the results.

TABLE 2
Influence of difference in distance between plant
cells and surface of culture liquid on cell culture

| | | Distance (μm) | |
|---|---|---|---|
| | Amount (ml) of | from liquid | Difference |
| Particle gun | liquid medium | surface | from control |
| Treated section | 2.0 | 1000 | A |
| Treated section | 2.25 | 1250 | A |
| Treated section | 2.50 | 1500 | C |
| Treated section | 2.75 | 1750 | B |
| Treated section | 3.0 | 2000 | C |
| Untreated section | 2.25 | 1225 | D |
| (control) | | | |

A: Significant influence,
B: Moderate influence,
C: Less influence,
D: No influence

After the introduction of the nucleic acid by the particle gun, the culture was continued, and changes in the cell were investigated. As a result, the cells continued to divide in all sections, but an obvious difference in the culture results was recognized in fertilized eggs in sections with the amount of the medium being 2.25 ml or less (the distance between the fertilized egg cells and the upper surface of the liquid phase was 1250 μm or less) as compared with the untreated section. Further, it was confirmed that there was a difference in morphology of culture also in the 3.0 ml section (the distance between the fertilized egg cells and the upper surface of the liquid phase was 2000 μm) as compared with the untreated section. It was inferred from these results that the gold particles had reached the cells in the liquid phase in the case where the distance between the cell and the surface of the culture liquid was short.

### Example 5 Nucleic Acid Introduction into Rice Cultured Cells (Oc Cells)

In this example, the nucleic acid was introduced into rice cultured cells.

The rice cultured cells (Oc cells) are a strain distributed from Riken BioResource Research Center. Cells subcultured in RIKEN and suspended in a liquid medium were used. The cells were taken out from the liquid medium in a flask, placed uniformly on a millicell membrane with a diameter of 1 cm, and subjected to a nucleic acid introduction test.

Gold particles with the GFP plasmid attached were injected using the particle gun into rice cultured cells (Oc cells) under the condition that the distance between the cells and the liquid surface was adjusted to 1250 μm based on the results of Example 4. The distance between the stopping screen and the sample was 6 cm to 9 cm. The nucleic acid was introduced by the particle gun method according to the method of Example 3, unless otherwise specified.

The cells were cultured for 3 days from the nucleic acid introduction and were thereafter observed with a fluorescence microscope. As a result, fluorescence due to GFP was observed as shown in FIG. 2. This result demonstrated that the GFP plasmid was introduced into the cells in the liquid phase.

### Example 6 Nucleic Acid Introduction into Maize Cultured Cells in Medium Containing Gelling Agent

In this example, the nucleic acid was introduced into maize cultured cells cultured in a medium containing a gelling agent.

The maize cells used were obtained by culturing the fertilized egg cells isolated in Example 2 in the dark at 26° C. overnight (for about 24 hours) and were a pre-embryonic cell group with the number of cells being about 1 to 2. The nucleic acid was introduced by the particle gun method according to the method of Example 3, unless otherwise specified.

The fertilized maize eggs were put into millicells, and the distance between the surface of the liquid medium and the fertilized eggs was adjusted to about 1000 μm to 1500 μm. Further, an experiment section in which a gelled liquid medium containing 0.3% agarose was put was set in the millicells, in order to stabilize the fertilized eggs in the culture liquid.

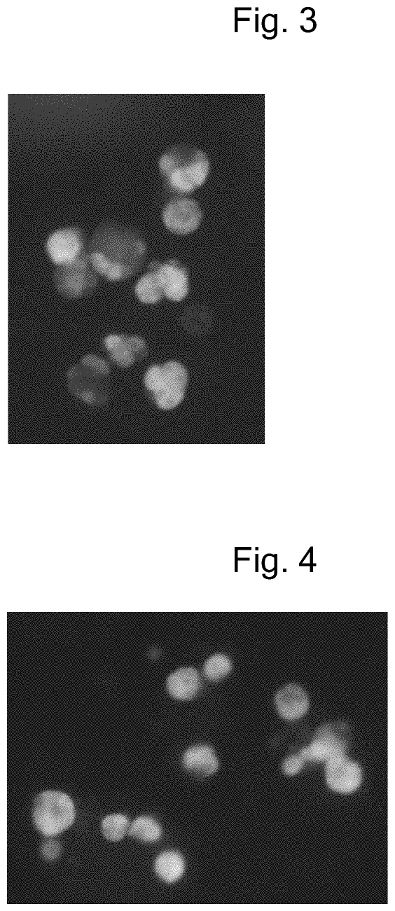

After the injection, the cells were continuously cultured for 3 days under the same conditions at the start of culture, and then the presence or absence of green fluorescence was checked using a fluorescence microscope. As a result, it was confirmed that the GFP plasmid was introduced with high efficiency (FIG. 3: the case of the culture medium containing gel, and FIG. 4: the case of the culture medium not containing gel). It is understood from FIGS. 3 and 4 that the GFP plasmid was introduced into the cells in the liquid phase whether the culture medium contains gel or not.

### Example 7 Culture of Cells with Substance Introduced

In this example, the maize cells with the nucleic acid introduced in Example 4 were further cultured.

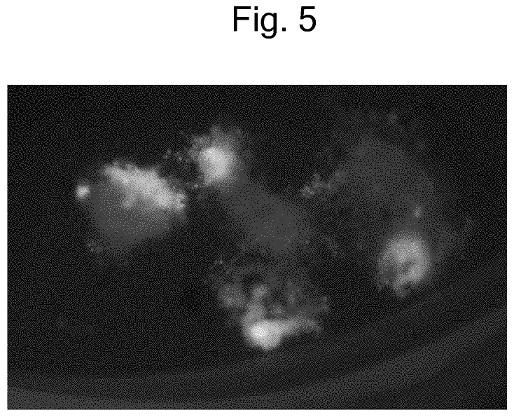

The nucleic acid-introduced cells obtained in Example 4 were continuously cultured under the same conditions at the start of culture. The fertilized eggs continued to divide after the introduction of the substance, and further fluorescence due to the GFP plasmid introduced was observed. Further, fluorescence due to the GFP plasmid could be observed in some cell masses derived from the fertilized maize eggs with the substance introduced, even after a lapse of 1 week from the introduction (FIG. 5). That is, it is inferred that part of the plasmid introduced was not transiently expressed but continuously expressed by being inserted into a chromosome.

### INDUSTRIAL APPLICABILITY

The present invention enables efficient substance introduction, transformation, and culture using fertilized egg cells or a cell group in the pre-embryonic stage of plants as a target. This enables transformants or genome-edited individuals of plants, which have been conventionally difficult to transform due to the difficulty of culture or the like and thus to which useful traits could not be given, to be easily and stably obtained with good reproducibility.

1. A method for introducing a substance into a plant cell, comprising introducing a substance into a plant cell present in a liquid phase using a particle gun method.

2. The method according to claim 1, wherein the plant cell is any one of a plant germ cell, a fertilized plant egg cell, and a pre-embryonic cell group.

3. The method according to claim 1, wherein the plant cell is a fertilized plant egg cell.

4. The method according to claim 1, wherein the plant cell is a pre-embryonic cell group with the number of cells being 8 or less.

5. The method according to claim 1, wherein the plant cell is a pre-embryonic cell group with the number of cells being 4 or less.

6. The method according to claim 1, wherein the plant cell is a pre-embryonic cell group with the number of cells being 2 or less.

7. The method according to claim 1, wherein the plant cell is a fertilized plant egg cell having a cell wall.

8. The method according to claim 1, wherein the distance between the plant cell and the upper surface of the liquid phase is more than 0 μm.

9. The method according to claim 1, wherein the distance between the plant cell and the upper surface of the liquid phase is 50 μm or more.

10. The method according to claim 1, wherein the distance between the plant cell and the upper surface of the liquid phase is 3000 μm or less.

11. The method according to claim 1, wherein the distance between the plant cell and the upper surface of the liquid phase is 2000 μm or less.

12. The method according to claim 1, wherein the substance to be introduced is selected from the group consisting of nucleic acids, proteins, peptides, polysaccharides, lipids, and cell organelles.

13. The method according to claim 1, wherein the liquid phase is a solution in a medium containing a gelling agent.

14. The method according to claim 1, wherein the plant cell is a cell of a monocotyledonous plant.

15. The method according to claim 14, wherein the plant cell is a cell of a gramineous plant.

16. The method according to claim 14, wherein the plant cell is a cell of a plant selected from the group consisting of maize, wheat, barley, rice, and sorghum.

17. The method according to claim 1, comprising the steps of:
(1) obtaining a plant cell that is any one of a plant germ cell, a fertilized plant egg cell, and a pre-embryonic cell group;
(2) culturing the plant cell obtained in step (1) in the liquid phase, wherein the distance is adjusted between the plant cell obtained in step (1) and the upper surface of the liquid phase; and
(3) introducing the substance into the plant cell present in the liquid phase using the particle gun method.

18. A substance-introduced plant cell obtained by the method according to claim 1.

19. A substance-introduced plant comprising the plant cell of claim 18.
